(19) United States
(12) Patent Application Publication
(10) Pub. No.: US 2008/0200538 A1
(43) Pub. Date: Aug. 21, 2008

(54) Inhibition of Monocyte Survival, Differentiation, or Proliferation

(75) Inventors: Andrea Doseff, Columbus, OH (US); Erich Grotewold, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation

(21) Appl. No.: 11/915,554

(22) PCT Filed: May 26, 2006
(86) PCT No.: PCT/US2006/020905

§ 371 Date: Nov. 26, 2007

Related U.S. Application Data
(60) Provisional application No. 60/684,655, filed on May 26, 2005.

(51) Int. Cl. A61K 31/352; A61P 29/00

(52) U.S. Cl. 514/456

(57) ABSTRACT

Methods comprising administering to the subject apigenin, an apigenin derivative, apigenin and at least one apigenin derivative, or a combination of apigenin derivatives are provided for treating inflammation in a subject in need of the same.

### CROSS-REFERENCE TO RELATED APPLICATIONS

This application claims priority to U.S. Provisional Application No. 60/684,655, filed May 26, 2005, which is incorporated herein by reference in its entirety.

### STATEMENT ON FEDERALLY FUNDED RESEARCH

Research leading to this invention was funded, at least in part by National Research Initiative of the USDA Cooperative State Research, Education and Extension Service, grant number 2002-35301-12028 and by the ACS-Ohio Division grant number GRT8355600. The government has certain rights in this invention.

### FIELD OF THE INVENTION

The invention relates to methods of inhibiting survival of stimulated or transformed monocytes and to methods of treating subjects with diseases associated with enhanced survival of stimulated monocytes, such as chronic inflammatory diseases, and/or enhanced proliferation of monocytes, such as acute monocytic leukemia.

### BACKGROUND OF THE INVENTION

Monocytes are produced in the bone marrow and constitute about 5% of the total white blood cells found in the circulation. Monocytes usually circulate in the bloodstream for 24-48 hours. In the absence of growth factors or transformation, circulating monocytes die by a mechanism known as apoptosis.

Monocytes defend mammals from pathogen (e.g. bacteria) infections. Monocytes that have been in contact with bacteria are stimulated. Monocytes respond to such stimulation by generating inflammatory mediators or cytokines (e.g. IL-8, IL-1β, TNFα, etc) and having a prolonged survival which leads to their accumulation at sites of inflammation. Monocytes are involved in many inflammatory diseases, particularly chronic inflammatory diseases. Inflammation is the general term for the local accumulation of fluid, plasma proteins and white blood cells that is initiated by physical injury, infection, or a local immune response. Acute inflammation is the term used to describe early and often transient episodes, while chronic inflammation occurs when an infection persists or during autoimmune responses.

Malignant transformed monocytes also exhibit prolonged survival. Transformed monocytes are involved in acute monocytic leukemia.

### SUMMARY OF THE INVENTION

Provided herein are methods of inhibiting the survival of monocytes, particularly stimulated and/or transformed monocytes. The method comprises contacting the monocytes with apigenin, a natural derivative of apigenin including, but not limited to, an apigenin glycoside, or a synthetic derivative of apigenin. Also, provided herein are methods of treating a subject with a disease associated with abnormal accumulation of monocytes, including, but not limited to, a chronic inflammatory diseases and acute monocytic leukemia. The method comprises treating the subject with apigenin, and/or one or more apigenin derivatives. As used herein the term “apigenin derivative” includes pharmaceutically acceptable salts of apigenin, a monocytic apoptosis-inducing metabolite of apigenin, a naturally-occurring derivative of apigenin, and a synthetic derivative of apigenin, or any combination of such compounds. Apigenin has the structure shown below.

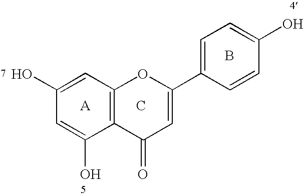

Suitable naturally occurring apigenin derivatives for use in the present methods include, but are not limited to C- and O-glycosylated apigenins such as the C-glycosyl flavones (e.g., maysin, isoorientin, and isovitexin) abundantly present in maize and other related plants. Suitable synthetic derivatives for use in the present methods are those in which the hydroxyl group attached to C-7 and/or C-5 in the A ring and/or the hydroxyl group attached to C-4′ in the B ring are glycosylated or acylated or replaced with amino group or halogens (e.g., Cl) and/or by the addition of nitro or amino groups at position 5′ in the B ring.

In one embodiment, the method comprises administering a therapeutically effective amount of apigenin and/or at least one apigenin derivative to subjects having an inflammatory disease or condition associated with stimulated or transformed monocytes. In certain embodiments, the method comprises administering a therapeutically effective amount of apigenin and/or at least one apigenin derivative to subjects having a chronic inflammatory disease, such as an autoimmune disease, arthritis, atherosclerosis, sarcoidosis or sepsis.

In certain embodiments, the method comprises administering a therapeutically effective amount of apigenin and/or one or more apigenin derivatives to a subject in need of the same, wherein the subject obtains a therapeutic benefit resulting from the administration of apigenin and/or the one or more apigenin derivatives.

Further provided are uses of apigenin and/or at least one apigenin derivative in the preparation of a medicament for treating inflammation in subjects, particularly mammalian subjects, and preferably human subjects. In certain embodiments, the use of apigenin or an apigenin derivative in a medicament is for treatment of a chronic inflammatory disease including, but not limited to, an autoimmune disease or arthritis.

Further provided are methods of using apigenin or a derivative thereof to suppress the differentiation of monocytes. In accordance with the present invention, the suppression of monocyte differentiation may occur either in vivo or in vitro.

Further provided are methods of using apigenin and/or at least one apigenin derivative to suppress the proliferation of monocytes. In accordance with the present invention, the suppression of monocyte proliferation may occur either in vivo or in vitro.

Further provided are methods of using apigenin and/or one or more apigenin derivatives to treat subjects with acute monocytic leukemia.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiments of the invention and together with the description, serve to explain the principles of the invention.

### BRIEF DESCRIPTION OF THE DRAWINGS

FIG. 1. Effect of apigenin and naringenin in cell survival of cancer cells. (A) Structure of the flavonoids apigenin and naringenin. (B) THP-1, U937, A549, and MCF-7 cells were treated with various doses of apigenin or naringenin for 24 h. After the treatment, the percentage of cell proliferation was calculated as the ratio of treated cells to control cells as determined by the MTT method (A490). Data represents means ±SEM (N=9).

FIG. 2. Apigenin induces cell death in monocytic leukemia cells. THP-1 and U937 leukemia cells were treated for various lengths of time with 50 μM apigenin or left untreated (NT) and stained with calcein AM and PI as described in Material and Methods to evaluate the percentage of cell survival. (A) THP cells after 12 h cells treated with 50 μM apigenin or with DMSO (NT). (B) The percentage of cell survival represented by means ±SEM (N=3).

FIG. 3. Apigenin induces caspase activation in monocytic leukemia. THP-1 (top) and U937 (bottom) leukemia cells were treated for various lengths of time with 50 μM apigenin or DMSO (NT). (A and C) Caspase-9 activity was determined by the LEHDAFC assay. (B and D) Caspase-3 activity was determined by the DEVD-AFC assay. Data represents means ±SEM (N=3).

FIG. 4. Caspase-3 activation is required for apigenin-induced apoptosis. THP-1 cells were treated for 12 h with 50 μM apigenin alone or pretreated with 20 μM DEVD-FMK for 1 h prior to the addition of apigenin. (A) Cells were then stained with calcein AM and 24 PI and the percentage of apoptotic cells was determined. (B) Lysates from cells treated as described above were used to determined caspase-3 activity by the DEVD-AFC assay. All data represents means ±SEM (N=5).

FIG. 5. Apigenin affects Akt phosphorylation. THP-1 cells were treated with 50 μM apigenin for different lengths of time. Lysates were separated by SDS-PAGE, transferred and immunoblotted with anti-phospho-Akt (pSer 473), anti-phospho-Akt (pThr 308), total Akt, and â-tubulin antibodies.

FIG. 6. Apigenin induces the activation of p38 and inactivation of Akt. A. THP-1 cells were treated with 50 μM apigenin for various lengths of time. Lysates were separated by SDS-PAGE, transferred and analyzed by immunoblots with anti-phospho-p38 (pp 38) and anti-total-p38 antibodies. B. THP-1 cells were treated for 6 hr with 50 μM apigenin alone (lane 2), treated with the apigenin diluent (lane 1), pretreatred with 10 or 25 μM SB203580 for 1 hr prior to the addition of apigenin (lanes 3 and 4) or with the SB203580 inhibitor alone (lanes 5 and 6). Lysates were analyzed by immunoblotting with anti-phospho-Akt (pSer 473), anti-phospho-p38 (p-p38), and anti-total-p38 antibodies.

FIG. 7. Apigenin induced p38 activation is not required for apoptosis. A. Cells were pretreated for 1 h prior to addition of 50 μM apigenin or DMSO (−) with 10 or 25 μM concentrations of SB203580 or DMSO (−) for 12 h. The percentage of apoptotic cells was determined by calcein AM with PI stained cells. Data represents means ±SEM (N=5 p>0.05). B. Lysates from the same treatments were used to determined caspase-3 activity by the DEVD-AFC assay. Data represents means ±SEM (N=5 p>0.05).

FIG. 8. Model of possible pathways of apigenin-induced-apoptosis. Left side illustrates apigenin targeting multiple upstream and downstream targets. Right side illustrates a model in which apigenin targets a protein or proteins downstream that act in a feedback loop in the regulation of the p38-Akt pathway.

FIG. 9 corresponds to Example 2 herein, and provides experimental results showing that apigenin includes cell death on LPS-treated monocytes.

FIG. 10 corresponds to Example 3 herein, and provides experimental results showing that apigenin reactivates caspase-3 on LPS-stimulated monocytes.

FIG. 11 corresponds to Example 4 herein and provides experimental results showing the effect of apigenin on IL-1β release.

FIG. 12 corresponds to Example 5 herein and provides experimental results showing that apigenin inhibits the expression of inflammatory cytokines.

### DETAILED DESCRIPTION OF THE INVENTION

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

It has now surprisingly been demonstrated that apigenin can induce cell death of LPS stimulated monocytes and thereby reduce the survival of LPS-stimulated monocytes in vitro.

Incubation of monocytes stimulated with LPS with an increasing amount of apigenin induces the death of such stimulated monocytes in an apigenin-dose dependent manner. Apigenin significantly increases caspase-3 activation in LPS stimulated monocytes, and it also inhibits the release of IL-1β, TNFα, IL-8 and the release of pro-inflammatory cytokines by LPS stimulated monocytes.

The invention relates to the use of apigenin and/or an apigenin derivative for treating an inflammatory condition or disease, particularly a chronic inflammatory condition or disease, in a subject in need of the same. In a certain embodiment of the invention the inflammatory diseases comprise autoimmune diseases, arthritis, and lung injuries.

The invention also relates to the use of apigenin and/or an apigenin derivative for treating acute monocytic leukemia in a subject in need of the same.

By “treating” is meant curing, ameliorating, reducing, or tempering the severity of the chronic inflammatory disease or acute monocytic leukemia, or the symptoms associated therewith. The terms “treating,” “treatment,” and “therapy” as used herein refer to curative therapy, prophylactic therapy, and preventative therapy.

The term “treating” shall be understood as referring to a subject obtaining any therapeutic benefit resulting from the administration of apigenin and/or at least one apigenin derivative, including a reduction of at least one symptom of the condition or conditions for which apigenin and/or the at least one apigenin derivative is administered, or inhibition or delay of the development or progression of the condition or conditions for which apigenin and/or the at least one apigenin derivative is administered.

The term “subject in need of treatment” shall be understood as referring to a mammal having at least one symptom, at least one risk factor, or a genetic predisposition for an inflammatory disease or condition, particularly a chronic inflammatory disease or condition and/or acute monocytic leukemia.

The term “therapeutically effective amount” shall be understood as referring to the amount of the compound or compounds of the present invention which, alone or in combination with other drugs, provides any therapeutic benefit in the prevention, treatment, or management of at least one of the symptoms, complications, or conditions associated with enhanced survival of monocytes including a chronic inflammatory disease or acute monocytic leukemia.

The terms “therapeutically effective” and “pharmacologically effective” are intended to qualify the amount of apigenin and/or apigenin derivative that, over absence of treatment, will achieve the goal of improvement in healing, particularly reducing inflammation, in a subject suffering from an inflammation. The apigenin and/or at least one apigenin derivative is useful in the treatment of chronic inflammatory diseases. The apigenin and/or apigenin derivative is also useful in the treatment of acute monocytic leukemia.

As used herein, “inflammation” and “inflammatory disease” refer to inflammation involved with, or causally related with monocytes. As used herein, “inflammation” and “inflammatory disease” encompass chronic inflammatory conditions. Some non-limiting examples of inflammation include coronary artery diseases, autoimmune diseases, arthritis, transplant-associated rejections, lung injuries, atherosclerosis, and pulmonary fibrosis. Apigenin and/or the at least one apigenin derivative may be used to alleviate inflammation in the subject as a short-term or long-term treatment, or may be prophylactic, as to suppress atherosclerosis or pulmonary fibrosis.

The term “subject” for purposes of treatment includes any mammalian subject who has experienced, is experiencing, or is at risk of developing a chronic inflammatory disease or condition or who has experienced, is experiencing, or is at risk of developing acute monocytic leukemia. In addition to being useful for human treatment, the compounds of the present invention are also useful for veterinary treatment of mammals, including companion animals and farm animals, such as, but not limited to dogs, cats, horses, cows, sheep, and pigs. Preferably, subject means a human.
Apigenin has the structure shown below:

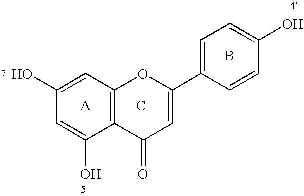

In one embodiment, the apigenin derivative is a pharmaceutically acceptable salt, ester, or monocyte apoptosis-inducing metabolite of apigenin. In another embodiment, the apigenin derivative is a naturally occurring derivative that has been isolated from a plant. Apigenin and naturally-occurring apigenin derivatives are found in many plants, including but not limited to maize. Examples of naturally-occurring apigenin derivatives include, but are not limited to maysin, isoorientin, and isovitexin. In certain embodiments the apigenin derivative is a synthetic molecule wherein the hydroxyl group attached to C-7 and/or C-5 in the A ring and/or the hydroxyl group attached to C-4′ in the B ring are glycosylated or acylated or replaced with amino group or halogens (e.g., Cl) and/or by the addition of nitro or amino groups at position 5′ in the B ring. Methods of synthesizing such synthetic derivatives are known in the art. Exemplary methods of making synthetic derivatives are described below.

Exemplary Methods of Preparing Apigenin Derivatives.

### Apigenin Derivatives

Acetylation of Apigenin

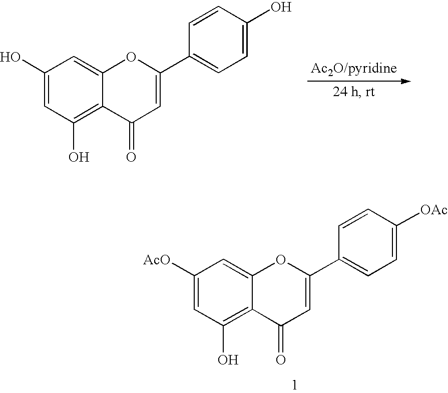

Acetic anhydride (2 eq) was added dropwise to a well stirred solution of apigenin in dry pyridine at ambient temperature under a nitrogen atmosphere. The solution was stirred for 24 h at room temperature and poured into ice-cold water. The precipitate (Compound 1) was filtered, dried and recrystallized from ethanol/acetone as a white solid [Al-Maharik N, Botting NP: Synthesis of lupiwighteone via a para-Claisen-Cope rearrangement. Tetrahedron 2003, 59(23):4177-4181.].
Methylation of Apigenin

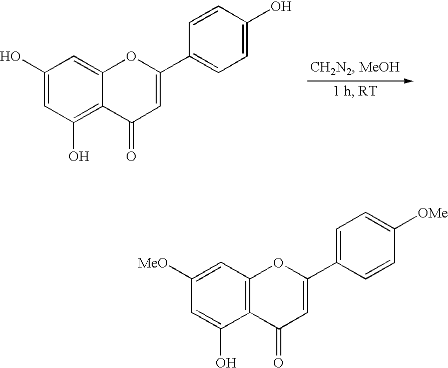

A solution of apigenin in MeOH was treated with ethereal diazomethane (CH₂N₂-Et₂O) until the yellow color persisted. The reaction solution was stirred at room temperature for 30 min. Removal of the solvent under reduced pressure furnished a residue, which was purified by silica gel column chromatography (2:1 hexanes/EtOAc) to give compound 2 [Matsuda H, Morikawa T, Toguchida I, Yoshikawa M: Structural requirements of flavonoids and related compounds for aldose reductase inhibitory activity. Chemical & Pharmaceutical Bulletin 2002, 50(6):788-795.].
Amination of Apigenin

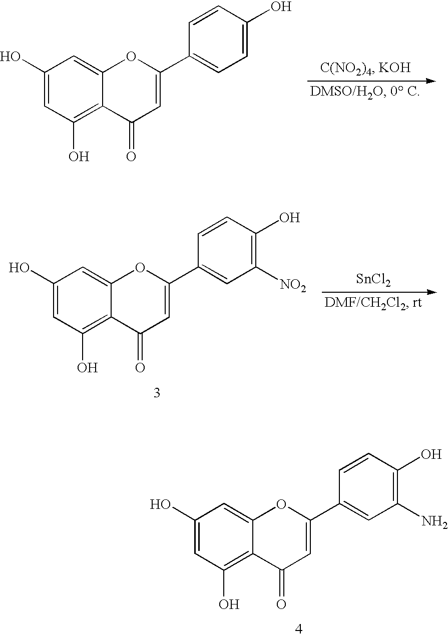

Apigenin was dissolved in a mixture of 0.11 N KOH solution and DMSO. To this solution, slowly add tetranitromethane (1.0 eq) at 5° C. The solution continued to stir for 2 hrs at 5° C. and then overnight at room temperature. After the reaction, the solution was acidified to pH<7 and the solvent was removed. The residue was redissolved and recrystallized to provide compound 3 [Bruice T C, Gregory M J, Walters S L: Reactions of tetranitromethane. I. Kinetics and mechanism of nitration of phenols by tetranitromethane. Journal of the American Chemical Society 1968, 90(6):1612-1619.].

Compound 3 and SnCl₂ (60 eq) were dissolved in a mixture of DMF and CH₂Cl₂ and stirred overnight under nitrogen at room temperature. After removal of the solvent, the residue was washed with KF solution completely, followed by water and brine. After filtration and recrystallization, compound 4 was obtained.

Pharmaceutical Compositions

Another aspect of the invention provides pharmaceutical compositions comprising an apigenin derivative, particularly a synthetic apigenin derivative, in combination with an acceptable carrier or excipient therefor and optionally with other therapeutically-active ingredients or inactive accessory ingredients. The carrier is pharmaceutically-acceptable in the sense of being compatible with the other ingredients of the formulation and not deleterious to the recipient. The pharmaceutical compositions include those suitable for oral, topical, inhalation, rectal or parenteral (including subcutaneous, intramuscular and intravenous) administration.

Formulations

Compositions are provided that contain therapeutically effective amounts of the apigenin-related compounds employed in the methods of the invention. The compounds can be formulated into suitable pharmaceutical preparations such as tablets, capsules, or elixirs for oral administration or in sterile solutions or suspensions for parenteral administration. The compounds described herein can be formulated into pharmaceutical compositions using techniques and procedures well known in the art.

The apigenin-related compound or mixture of apigenin compounds is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage is obtained. The compositions can be formulated in a unit dosage form. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, the apigenin-related compounds employed in the methods of the invention are mixed with a suitable pharmaceutically acceptable carrier. Upon mixing or addition of the compound(s), the resulting mixture may be a solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

When the compounds exhibit insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The apigenin-related compounds employed in the methods of the invention may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, an inventive compound in lyophilized form and a suitable diluent may be provided as separated components for combination prior to use. A kit may include an inventive compound and a second therapeutic agent for co-administration. The inventive compound and second therapeutic agent may be provided as separate component parts. A kit may include a plurality of containers, each container holding one or more unit dose of the inventive compound employed in the method of the invention. The containers can be adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active inventive compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Formulations of the present invention suitable for oral administration may be presented as discrete units such as capsules, cachets, tablets, boluses or lozenges, each containing a predetermined amount of the active compound; as a powder or granules; or in liquid form, e.g., as an aqueous solution, suspension, syrup, elixir, emulsion, dispersion, or the like.

Formulations suitable for parenteral administration conveniently comprise a sterile preparation of the active compound in, for example, water for injection, saline, a polyethylene glycol solution and the like, which is preferably isotonic with the blood of the recipient.

Useful formulations also comprise concentrated solutions or solids containing apigenin and/or one or more apigenin derivatives, which upon dilution with an appropriate solvent give a solution suitable for parenteral administration.

Preparations for topical or local applications comprise aerosol sprays, lotions, gels, ointments, suppositories etc., and pharmaceutically-acceptable vehicles therefore such as water, saline, lower aliphatic alcohols, polyglycerols such as glycerol, polyethylene glycerol, esters of fatty acids, oils and fats, silicones, and other conventional topical carriers. In topical formulations, the subject compounds are preferably utilized at a concentration of from about 0.1% to 5.0% by weight.

In addition to the aforementioned ingredients, the formulations of this invention may further include one or more optional accessory ingredient(s) utilized in the art of pharmaceutical formulations, i.e., diluents, buffers, flavoring agents, colorants, binders, surface active agents, thickeners, lubricants, suspending agents, preservatives (including antioxidants) and the like.

Modes of Administration

In one embodiment, the mode of administration of apigenin and/or the one or more apigenin derivatives will be oral. In other embodiments, the mode of administration is parenteral, intradermal, subcutaneous or topical. In certain embodiments, e.g. when the subject has arthritis, apigenin and/or the apigenin derivative is administered as a topical or local application. In certain embodiments, e.g., when the subject has leukemia, the active ingredients are administered intravenously or orally. In other embodiments, e.g. when the subject has sarcoidosis, administration is by inhalation.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art.

The apigenin-related compounds used in the present methods may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

Compounds employed in the methods of the invention may be administered enterally or parenterally. When administered orally, compounds employed in the methods of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods of the invention need to be administered only once or twice daily.

The oral dosage forms can be administered to the patient 1, 2, 3, 4, or more times daily. The inventive compounds employed in the methods of the invention can be administered either three or fewer times, or even once or twice daily. Hence, the inventive compounds employed in the methods of the invention can be administered in oral dosage form. Whatever oral dosage form is used, they can be designed so as to protect the compounds employed in the methods of the invention from the acidic environment of the stomach. Enteric coated tablets are well known to those skilled in the art. In addition, capsules filled with small spheres each coated to protect from the acidic stomach, are also well known to those skilled in the art.

Dosage

The composition comprising apigenin and/or one or more apigenin derivatives is administered to the subject in a therapeutically effective amount. The dosages of the compounds needed to obtain a therapeutic effect can be determined in view of this disclosure by one of ordinary skill in the art by running routine trials with appropriate controls. Comparison of the appropriate treatment groups to the controls will indicate whether a particular dosage is therapeutically effective.

The amount of the compositions of the present invention required will depend upon the nature and severity of the condition being treated, and on the nature of prior treatments which the subject has undergone. Ultimately, the dosage will be determined using clinical trials. Initially, the clinician will administer doses that have been derived from animal studies. An effective amount can be achieved by one administration of the composition. Alternatively, an effective amount is achieved by multiple administration of the composition to the subject. In vitro, the biologically effective amount, i.e., the amount sufficient to induce glucose uptake, is administered in two-fold increments, to determine the full range of activity. The efficacy of oral, subcutaneous and intravenous administration is determined in clinical studies. Although a single administration of the compositions may be beneficial, multiple doses may also be beneficial.

Depending on species, age, individual condition, mode of administration and the clinical picture in question, effective doses of apigenin, for example, corresponding to daily doses of the active substance (free base) of about 10-1000 mg, preferably 50-600 mg, especially 100400 mg, are administered to warm-blooded animals of about 70 kg bodyweight. For adult patients with inflammatory diseases a starting dose of, e.g., 200 mg daily can be recommended. For patients with an inadequate response after an assessment of response to therapy with 200 mg daily, dose escalation can be safely considered and patients may be treated as long as they benefit from treatment and in the absence of limiting toxicities.

The invention may be better understood by reference to the following examples, which serve to illustrate but not to limit the present invention.

### EXAMPLES

### Example 1

### Treatment of Acute Monocytic Leukemia

We have investigated the effects of apigenin and naringenin on several cancer cells. The flavone apigenin is overall more effective than the flavanone naringenin in inhibiting cell proliferation and effectively induces apoptosis of THP-1 and U937 monocytic leukemia cells. Apigenin induces caspase-9 and caspase-3 activities in these two cell lines with distinct kinetics. Caspase activation is essential for the observed cell death, evidenced by the effect of the caspas-3 inhibitor in blocking apigenin-induced apoptosis. Apigenin treatment of THP-1 was accompanied by the rapid dephosphorylation of the PDK2-dependent-site (Ser473) in Akt, followed by the disappearance of the Akt protein. Apigenin also induced the activation of the p38 mitogen-activated protein kinase (MAPK). Pharmacological inhibition of p38 with the p38 inhibitor SB203580 showed that the apigenin-induced activation of p38 occurs upstream of Akt. Finally, inhibition of p38 failed to block apoptosis and caspase activation in apigenin treated cells, suggesting that p38 is not essential for the induction of the apoptotic pathway.

Introduction

Flavonoids are ubiquitous phenolic compounds broadly distributed in fruits and vegetables (Stafford, H. A. (1990) Flavonoid metabolism. Boca Raton, USA: CRC Press, Inc.). Depending on the organization of their cyclic benzene rings and their modifications, flavonoids can be classified into various groups that include flavan-3-ols, flavones, isoflavones, flavanones, and flavonols.

Apoptosis, or programmed cell death, plays a crucial role in normal development, homeostasis, and defense against pathogens (Doseff, A. I. (2004) Apoptosis: the sculptor of development. Stem Cells Developm., 13, 473-483). Essential executioners of apoptosis are the caspases, a family of conserved cysteine proteases (Thornberry, N. A. and Lazebnik Y. (1998) Caspases: enemies within. Science, 281, 1312-1316). The caspases are expressed as inactive precursors that become activated by apoptotic signals. Initiator caspases, such as caspase-9, receive the apoptotic signal and initiate the activation of caspase-3, an executioner caspase responsible for cleaving many cellular proteins during apoptosis (Cohen, G. M. (1997) Caspases: the executioners of apoptosis. Biochem. J, 326, 1-16). Apoptosis is characterized by several distinct morphological changes, which include nuclear condensation and fragmentation, cytoskeleton disruption, cell shrinkage, and membrane blebbing, which then lead to the formation of apoptotic bodies, recognized and engulfed by macrophages (White, E. (1996) Life, death, and the pursuit of apoptosis. Genes Dev., 10, 1-15, Platt, N., da Silva R. P. and Gordon S. (1998) Recognizing death: the phagocytosis of apoptotic cells. Trends Cell Biol., 8, 365-372). Defects of the apoptotic machinery have been implicated in the pathogenesis of cancer (Lowe, S. W., Ruley H. E., Jacks T. and Housman D. E. (1993) p53-dependent apoptosis modulates the cytotoxicity of anticancer agents. Cell, 74, 957-967). Monocytic leukemias arise by the malignant transformation of granulocytes or monocytes, blood cells responsible for the innate response to infectious pathogens. Monocytes normally undergo spontaneous apoptosis through a mechanism that requires caspase-3 (Fahy, R. J., Doseff A. I. and Wewers M. D. (1999) Spontaneous human monocyte apoptosis utilizes a caspase-3-dependent pathway that is blocked by endotoxin and is independent of caspase-1. J Immunol., 163, 1755-1762). In the presence of inflammatory or differentiation signals, monocytes escape their apoptotic fate and survive longer (Kelley, T. W., Graham M. M., Doseff A. I., Pomerantz R. W., Lau S. M., Ostrowski M. C., Franke T. F. and Marsh C. B. (1999) Macrophage colony-stimulating factor promotes cell survival through Akt/protein kinase B. J. Biol. Chem., 274, 26393-26398, Goyal, A., Wang Y., Graham M. M., Doseff A. I., Bhatt N. Y. and Marsh C. B. (2002) Monocyte survival factors induce AKT activation and suppress caspase-3. Am. J. Respir. Cell Mol. Biol., 26, 224-230). Similarly, upon malignant transformation, cells from the monocytic lineage undergo active proliferation characterized by the clonal expansion and the inhibition of the apoptotic program. Acute myelogenous leukemia (AML) is the most common type of leukemia in adults, with an estimated 10,000 or more new cases reported each year. Current therapies for leukemia include the treatment with chemotherapeutic drugs to induce death of cancer cells and, in the absence of incomplete remission, blood stem cells transplant. Thus, the search for alternative anti-cancer drugs to eliminate leukemia is an area of active research.

Prolonged survival of cancer cells is characterized by the activation of the serine/threonine kinase Akt/PKB (Toker, A. (1998) Signaling through protein kinase C. Front. Biosci., 3, d1134-d1147), generally considered to play a pro-survival function. Akt activation requires its phosphorylation at Thr308 by PDK1 (phosphatidylinositol-dependent-kinase) via the phosphoinositide-3-kinase (PI3-K) pathway (Alessi, D. R., Andjelkovic M. A., Caudwell B., Cron P., Morrice N., Cohen P. and Hemmings B. A. (1996) Mechanisms of activation of protein kinase B by insulin IGF-1. EMBO J, 15, 6541-6551) and the phosphorylation at Ser473 by a PDK2, the identity of which is believed to depend on the specific survival signals present (Partovian, C. and Simons M. (2004) Regulation of protein kinase B/AKT activity and Ser 473 phosphorylation by protein kinase c alpha in endothelial cells. Cell. Signaling, 16, 951-957, Anter, E., Thomas S. R., Schulz E., Shapira O. M., Vita J. A. and Keaney J. F., Jr. (2004) Activation of endothelial nitric-oxide synthase by the p38 MAPK in response to black tea polyphenols. J. Biol. Chem., 279, 46637-46643). The induction of apoptosis has also been associated with the activation of members of the mitogenactivated protein kinase (MAPK) family, which include p38, JNK, and ERK (Olson, J. M. and Hallahan A. R. (2004) p38 MAP kinase: a convergence point in cancer therapy. Trends Mol. Med., 10, 125-129). However, the requirement of p38 activation during apoptosis has been controversial, as the treatment with p38 inhibitors fails to inhibit apoptosis in some systems, while blocking apoptosis in others (Frasch, S. C., Nick J. A., Fadok V. A., Bratton D. L., Worthen G. S. and Henson P. M. (1998) p38 mitogen-activated protein kianse-dependent and -independent intracellular signal transduction pathways leading to apoptosis in human neutrophils. J. Biol. Chem., 273, 8389-8397). In addition, the relative position of the MAPKs, particularly of p38, with respect to Akt is unclear, with some studies suggesting that p38 is upstream of Akt (Anter, E., Thomas S. R., Schulz E., Shapira O. M., Vita J. A. and Keaney J. F., Jr. (2004) Activation of endothelial nitric-oxide synthase by the p38 MAPI in response to black tea polyphenols. J. Biol. Chem., 279, 46637-4664) and others proposing the opposite (Liao, Y. and Hung M. C. (2003) Regulation of the activity of p38 mitogen-activated protein kinase by Akt in cancer and adenoviral protein E1A-mediated sensitization to apoptosis. Mol. Cell. Biol., 23, 6836-6848).

Here, we describe the differential effect of apigenin and naringenin in their ability to induce apoptosis of the myeloblastic leukemia cell lines U937 and THP-1. We established that apigenin is a potent inducer of apoptosis in these leukemia cells, and that the activation of caspase-9 and caspase-3 is essential in this process. We also show that the p38 MAPK is activated during the apoptotic process, but that cell death proceeds independently of p38 activity. In addition, we show that apigenin has a dual effect on Akt. At short times, it promotes the dephosphorylation of Ser473 and at longer times induces the overall decrease of the Akt protein. Together, these studies provide evidence of the chemotherapeutic potential of apigenin for the treatment of myeloblastic leukemias and uncover novel aspects of the signal transduction components necessary for the observed apoptotic effect.

Materials and Methods

Materials and Cell Culture

All cells were grown at 37° C. in a humidified atmosphere of 95% air and 5% CO2 in media supplemented with 100 U/ml penicillin, and 100 μg/ml streptomycin (BioWhittaker). THP-1 and U937 cells were maintained in RPMI 1640 medium with Lglutamine (BioWhittaker, Walkersville, Md.) supplemented with 5% fetal bovine serum (FBS) (Hyclone, Logan, Utah) while A549 cells were supplemented with 10% FBS. MCF-7 cells were maintained in DMEM low glucose (Gibco) with 5% FBS. The flavonoids apigenin and naringenin, and the diluent dimethyl sulfoxide (DMSO) were obtained from Sigma-Aldrich (St. Louis, Mo.). The caspase inhibitor DEVD-FMK was obtained from Enzyme System Products (Livermore, Calif.). The p38 inhibitor SB203580 was obtained from Calbiochem (San Diego, Calif.).

Cell Viability Assay

Cell viability was assayed with CellTiter 96 Aqueous One Solution Cell Proliferation Assay as suggested by manufacturer (Promega, Madison, Wis.). Cells were plated at a density of 2×104 cells/well into 96-well plates and treated with flavonoids at indicated concentrations for 24 h. Absorbance at 490 nm (A490) was recorded using an ELISA plate reader (Bio-Tek ELx800, KC Junior, Winooski, Vt.).

Assessment of Cell Survival and Apoptosis

To investigate apoptosis, cells were plated at a density of 1×106 cells/well. After treatment, cells were collected and washed in PBS. Cells were then incubated in RPMI (no phenol red) and 1 μg/ml calcein AM for 30 min and 5×10−2 ng/ml propidium iodide (PI) for 5 min. Cells were washed twice and resuspended in PBS. Cells were viewed using a fluorescent microscope (Olympus, Melville, N.Y.). At least 200 cells were counted. Cells calcein AM positive (green) in the absence of PI (red) were considered alive while cells undergoing apoptosis are green with red. Number of green cells or green with red cells was counted over total number of cells (green alone and green with red) to express cell survival or apoptotic cell percentage respectively.

Measurement of Caspase Activity

For measurements of caspase activity, cells were plated at a density of 1.5×106 cells/well. After treatment, cells extracts were prepared as previously described (Doseff, A. I., Baker J. H., Bourgeois T. A. and Wewers M. D. (2003) Interleukin-4-induced zpoptosis entails caspase activation and suppression of extracellular signal-regulated kinase phosphorylation. Am. J. Resp. Cell Mol. Biol., 29, 367-374). Protein extracts were incubated with 20 μM DEVD-AFC to determine caspase-3 activity or LEHD-AFC to determine caspase-9 activity (Enzyme Systems Products, Livermore, Calif.) in a cytobuffer as previously described (Doseff, A. I., Baker J. H., Bourgeois T. A. and Wewers M. D. (2003) Interleukin-4-induced zpoptosis entails caspase activation and suppression of extracellular signal-regulated kinase phosphorylation. Am. J. Resp. Cell Mol. Biol., 29, 367-374). Levels of released AFC were measured using Cytofluor 400 fluorimeter (Filters: excitation 400 nm, emission 508 nm; Perspective Co., Framingham, Mass.).

Protein Analysis by Western Blot

Extracts from 3×106 cells were prepared by incubating cells for 30 min on ice in lysis buffer (50 mM Tris, 10 mM EDTA 0.5% NP-40, 10 mM Na-glycerophosphate, 5 mM Na-pyrophosphate, 50 mM NaF, 1 mM orthovanadate, 1 mM DTT, 0.1 mM PMSF, 2 μg/ml of protease inhibitors: chymostatin, pepstatin, antipain, and leupeptin). Cell lysates were centrifuged (14,000×g for 10 min at 4° C.) and the supernatants were stored for at −70° C. for future analysis. Equal amounts of protein were loaded and separated by SDS-PAGE, transferred onto nitrocellulose membranes and probed with antibodies of interest followed by horseradish peroxidase conjugated secondary antibody and visualized by enhanced chemiluminescence (Amersham, Arlington Heights, Ill.). Phospho-Ser473-Akt, phospho-Thr308-Alt, total AKT, phospho-p38 and total p38 antibodies were obtained from Cell Signaling (Boston, Mass.). â-tubulin antibody was obtained from Upstate (Charlottesville, Va.).

Statistical Analysis

All data are expressed as mean ±SEM and student t-test comparisons were conducted to analyzed statistical significance. Statistical significance is stated in the text.

Results

Apigenin Inhibited the Proliferation of Monocytic Leukemia Cells

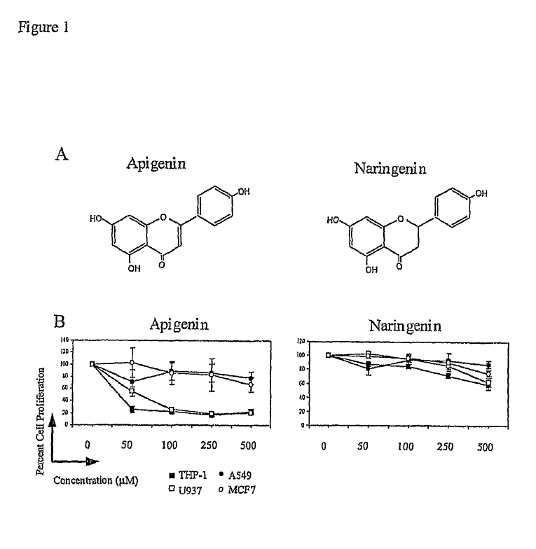

Apigenin and naringenin are structurally related flavonoids (FIG. 1A) that exert anti-proliferation properties (Harmon, A. W. and Patel Y. M. (2004) Naringenin inhibits glucose uptake in MCF-7 breast cancer cells: a mechanism for impaired cellular proliferation. Breast Cancer Res. and Treat., 85, 103-110, Way, T. D., Kao M. C. and Lin J. K, (2004) Apigenin induces apoptosis through proteasomal degradation of HER2/neu in HER2/neu-overexpressing breast cancer cells via the phosphatidylinositol 3-kinase/Akt-dependent pathway. J. Biol. Chem, 279, 4479-89). We first investigated the effect of these flavonoids in the proliferation of different cancer cell lines including the human monocytic leukemia THP-1 and U937 cell lines, the lung epithelial cell line A549, and the breast epithelial cell line MCF7. Treatment with 50 to 500 μM apigenin for 24 h reduced cell proliferation to approximately 20% in THP-1 cells (FIG. 1B). Treatment of U937 with 50 μM apigenin reduced cell proliferation to 50% while higher concentrations of apigenin reduced U937 cell proliferation to 20% (FIG. 1B). Apigenin-treatment of MCF-7 and A549 reduced cell proliferation to 80% (FIG. 1B). The treatment of these cells for up to 48 h with apigenin resulted in similar levels of survival (data not shown), suggesting that the maximum effect of apigenin is already obtained at 24 h. Treatment with up to 500 μM of naringenin reduced cell proliferation in MCF-7 and A549 cells only by 20% (FIG. 1B), while cell proliferation of THP-1 and U937 monocytic leukemia was reduced to 60% with 500 μM naringenin (FIG. 1B). These results together show that apigenin is a more potent flavonoid in inhibiting proliferation of cancer cells and this effect is more pronounced in monocytic leukemia cells, compared to epithelial cell lines.

Apigenin-Induced-Cell Death is Mediated by a Caspase-Dependent Pathway

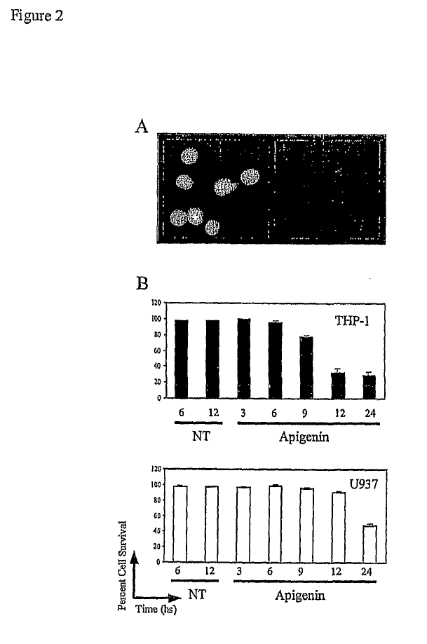

To establish whether the anti-proliferative activity of apigenin in the monocytic leukemia THP-1 and U937 cell lines was associated with the induction of cell death, we determined the number of apoptotic cells after the treatment of THP-1 and U937 with 50 μM apigenin for different lengths of time using the calcein AM/PI viability assay (FIG. 2A). Cells that display green fluorescence (calcein AM) in the absence of red (PI) are alive, while cells undergoing apoptosis are visualized by the combination of green and red. Using this method, we established that survival of THP-1 cells was reduced to 80% at 9 h and that cell viability decreased to 30% after the 12 h treatment with apigenin (FIG. 2B). U937 cells showed a minor decrease (10%) in cell survival after 12 h and a further decrease (50%) after 24 h treatment with apigenin (FIG. 2B).

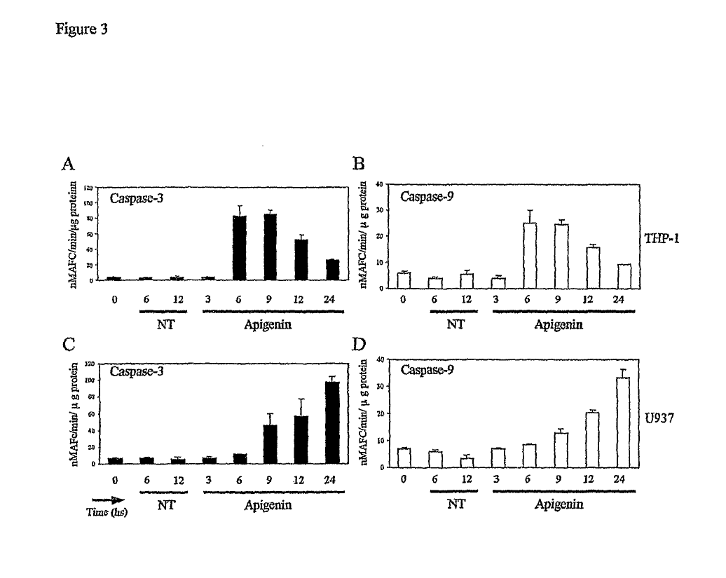

To determine whether apoptosis was involved in apigenin-induced cell death, we first studied the effect of apigenin on caspase activation. For this purpose, THP-1 and U937 cells were treated with 50 μM apigenin for various lengths of time and caspase-9 and caspase-3 activities were measured using the fluorogenic substrates LEHD-AFC and DEVD-AFC, respectively. In THP-1 cells, caspase-9 activity was observed after 6 h of treatment with apigenin and remained high after 9 h of treatment, decreasing after 12 h (FIG. 3A). Caspase-3 activity was detected after 6 h and similarly to caspase-9, the activity was sustained at 9 h, but decreased after 12 h of treatment with apigenin (FIG. 3B). In U937 cells, caspase-9 and caspase-3 activities were detected at 9 h after the treatment with apigenin, and the activities remained high, even after 24 h of the addition of apigenin (FIGS. 3C and D). These results suggest a distinct kinetic response of these two monocytic leukemia cell lines to the potent effect of apigenin.

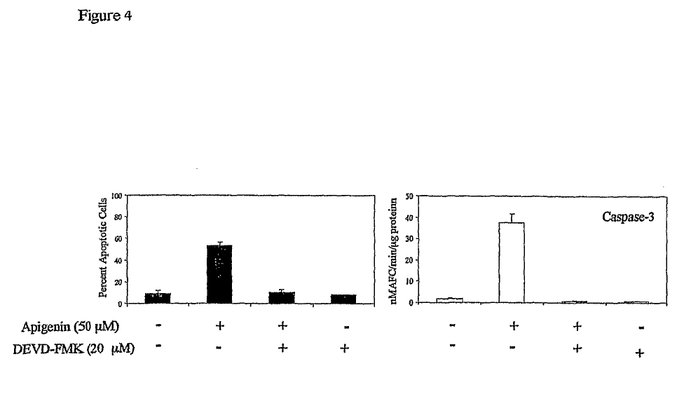

We next determined whether caspase-3 activity was required for apigenin induced cell death. THP-1 cells were incubated with the caspase-3 inhibitor DEVDFMK at 20 μM for 1 h prior to the addition of 50 μM apigenin for 12 h. Subsequently, the number of apoptotic cells and the activity of caspase-3 were assessed using the methods described above. We observed 70% of apoptotic cells after the treatment with apigenin, while the pre-treatment with the caspase inhibitor DEVD-FMK reduced the number of apoptotic cells to less of 10% (FIG. 4A). A similar percentage of apoptotic cells was observed in untreated or cells treated with DEVD-FMK alone (FIG. 4A). Consistent with these findings and further highlighting the central role of caspase-3 in this apoptotic process, we found that the pre-treatment with DEVD-FMK inhibited the apigenin-induced activation of caspase-3 to the levels observed in untreated cells (FIG. 4B). These results demonstrate that apigenin induces apoptosis of THP-1 and U937 leukemia cell lines though a caspase-9/caspase-3 mediated pathway.

Apigenin Inhibits Akt Activity in Monocytic Cells

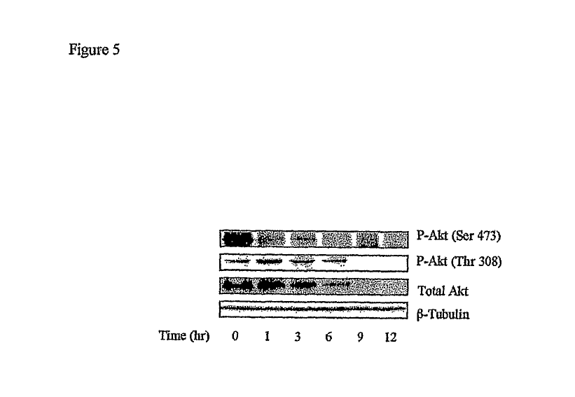

Activation of Akt is believed to provide an important survival signal. To examine the mechanisms involved in apigenin-induced-apoptosis, we characterized the effect of apigenin on Akt phosphorylation. THP-1 cells were treated with 50 μM apigenin for different lengths of time and lysates were assayed for the presence of activated Alit by Western blot analyses. Using an anti-Akt polyclonal antibody that detects Akt when it is phosphorylated at Ser473 (FIG. 5, pSer473), the PDK2 site, we observed that exposure of THP-1 cells to apigenin induced a rapid decrease in Alt pSer473 phosphorylation during the first hour (FIG. 5). At this time, the levels of Akt protein remain unchanged, as evidenced by the total Akt levels detected by a polyclonal antibody (FIG. 5, Total Akt). We also investigated the phosphorylation of Thr308 (FIG. 5, pThr308), the site phosphorylated in a PDK1-PI3-K-mediated (Stokoe, D., Stephens L. R., Copeland T., Gaffney P. R., Reese C. B., Painter G. F., Holmes A. B., McCorrnick F. and Hawkins P. T. (1997) Dual role of phosphatidylinositol-3,4,5-trisphosphate in the activation of protein kinase B. Science, 277, 567-570), using an antibody that specifically recognizes Akt pThr308. Interestingly, the phosphorylation at this site was not significantly affected during the first six hours of apigenin treatment. After this time, the levels of total Akt dramatically decreased. These results indicate that apigenin affects Akt by two mechanisms: First, it decreases the phosphorylation of the PDK2 site and second, induces a decrease in the total Akt protein levels.

Akt Inactivation by Apigenin Requires Activation of p38

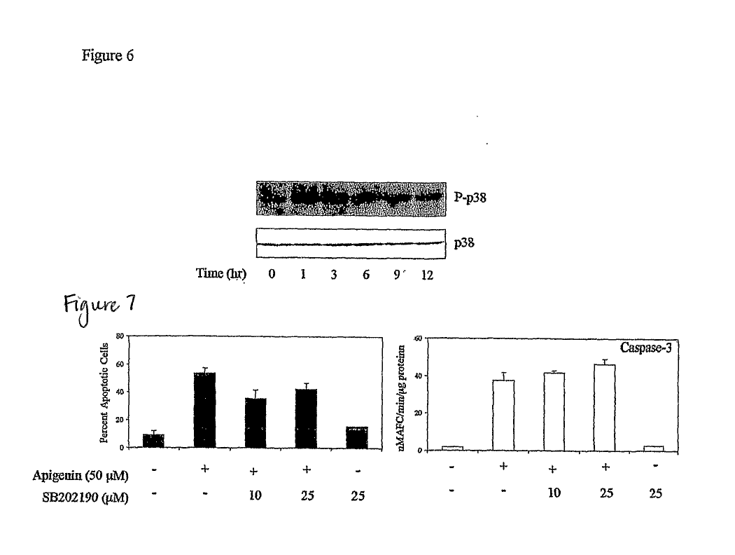

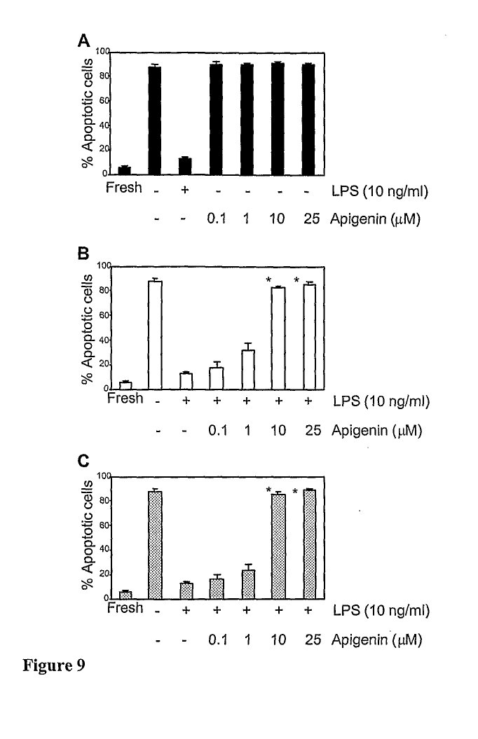

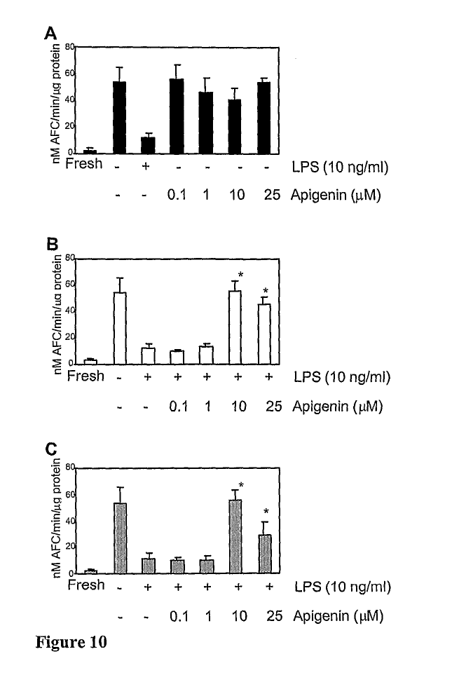

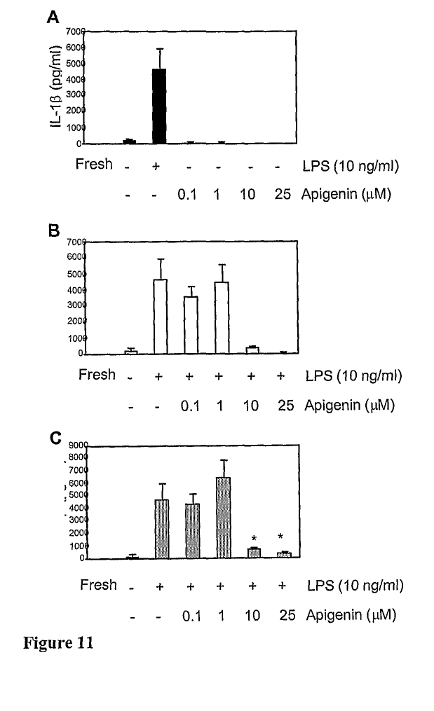

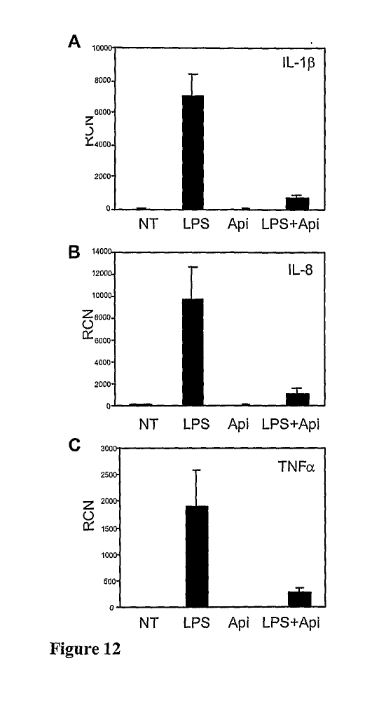

Because the activation of the stress-induced MAPK p38 has been observed in several cell types treated with other phenolic compounds of plant origin (Anter, E., Thomas S. R., Schulz E., Shapira O. M., Vita J. A. and Keaney J. F., Jr. (2004) Activation of endothelial nitric-oxide synthase by the p38 MAPK in response to black tea polyphenols. J. Biol. Chem., 279, 46637-46643), we next examined the effect of apigenin on the activity of p38 in monocytic leukemia. THP-1 cells were treated with 50 μM apigenin for various lengths of time, or left untreated, and the phosphorylation of p38 was investigated by Western blotting using an anti-phospho p38 antibody. An increase in the phosphorylation of p38 (FIG. 6A, p-p38) was observed after 3 h of treatment with apigenin.

We next examined the relation between Akt and p38 during the apigenin-induced apoptosis. THP-1 cells were pretreated for 1 h with 10 or 25 μM of the p38 phosphorylation inhibitor SB203580. After that period of time, cells were treated with 50 μM apigenin for 3 h and the activation of Akt and p38 was determined by immunoblotting. We found that in cells pretreated with SB203580, the apigenin-induced p38 phosphorylation was significantly reduced, that was accompanied by an increase of Akt phosphorylation at Ser473 (FIG. 6B, lanes 3 and 4).

We next investigated whether the activation of p38 was required for apigenin induced cell death in THP-1 cells. For this purpose, we compared the number of apoptotic cells in THP-1 cultures treated with 50 μM apigenin alone, treated with the p38 inhibitor SB203580 for 1 h prior to the addition of apigenin or cells left untreated. We found that the treatment with SB203580 did not result in a reduction of the number of apoptotic cells induced by apigenin (FIG. 7A). We found a similar percentage of apoptotic cells in apigenin and SB203580-treated cultures, compared to cells treated with apigenin alone (FIG. 7A, no statistical difference P>0.05, Student s t-test). Consistent with this finding, we found that caspase-3 activity was similar in cells treated with both SB203580 and apigenin as in cells treated with apigenin alone (FIG. 7B, no statistical difference P>0.05, Student s t-test). These results, taken together, suggest that the activation of p38 is induced by apigenin but is not essential for the execution of apoptosis.

Discussion

Flavonoids are emerging as potent cancer prevention and chemotherapeutic agents. Previous studies have shown that apigenin induces cell death to some extent in human colon carcinoma cell lines, breast epithelial cells, and in lymphocytic leukemia cells (Way, T. D., Kao M. C. and Lin J. K. (2004) Apigenin induces apoptosis through proteasomal degradation of HER2/neu in HER2/neu-overexpressing breast cancer cells via the phosphatidylinositol 3-kinase/Akt-dependent pathway. J. Biol. Chem, 279, 4479-89, Wang, W., Heideman L., Chung C. S., Pelling J. C., Koehler K. J. and Birt D. F. (2000) Cell-cycle arrest at G2/M and growth inhibition by apigenin in human colon carcinoma cell lines. Mol. Carcinog., 28, 102-110, Wang, I.-K., Lin-Shiau S. Y. and Lin J. K. (1999) Induction of apoptosis by apigenin and related flavonoids through cytochrome C release and activation of caspase-9 and caspase-3 in leukaemia HL-60 cells. Europ. J Cancer, 35, 1517-1525). Our results expand these studies demonstrating that apigenin is particularly effective in inducing apoptosis of the THP-1 and U937 myeloblastic leukemia cells. We determined that the flavone apigenin induces apoptosis much more effectively than the related flavanone, naringenin (FIG. 1). Previous studies showed that apigenin was more potent in its ability to induce apoptosis of HL-60 lymphoblastic leukemia cells than the flavonols kaempferol, and quercetin, leading to the suggestion that the absence of the 3-hydroxyl group (C ring) is in part responsible for its potency (Wang, I.-K., Lin-Shiau S. Y. and Lin J. K. (1999) Induction of apoptosis by apigenin and related flavonoids through cytochrome C release and activation of caspase-9 and caspase-3 in leukaemia HL-60 cells. Europ. J Cancer, 35, 1517-1525). Our results suggest that this is probably not the case, since neither apigenin nor naringenin have a 3-hydroxyl group, yet display significant differences in their ability to induce apoptosis of THP-1 and U937 cells (FIG. 1B). More likely, it is the planar structure of apigenin, conferred by the double bond between carbons 2 and 3, which is responsible for the observed difference in potency. Reverse-phase high performance liquid chromatography experiments carried out with extracts of THP-1 cells treated with naringenin or apigenin showed that neither one of these two compounds is significantly converted to another chemical entity that could be responsible for the observed apoptotic activity (data not shown).

The p38 MAPK is involved in the regulation of a number of cellular responses to stress and its activation is necessary for the induction of cell death of cancer cells by a number of anti-cancer agents (Olson, J. M. and Hallahan A. R. (2004) p38 MAP kinase: a convergence point in cancer therapy. Trends Mol. Med., 10, 125-129). Consistent with these findings, apigenin treatment of THP-1 cells does result in an increased p38 phosphorylation (FIG. 6A). In contrast to p38 MAPK, the PI3K/Akt pathway is considered pro-survival. Consistent with the expected down-regulation of Akt for the induction of apoptosis, we observed a significant decrease in the phosphorylation of Ser473 immediately after apigenin addition (FIG. 6B). Constitutive phosphorylation of Ser473 in Akt has been associated with poor prognosis in patients with AML (Min, Y. H., Eom J. I., Cheong J. W., Maeng H. O., Kim J. Y., Jeung H. K., Lee S. T., Lee M. H., Hahn J. S. and Ko Y. W. (2003) Constitutive phosphorylation of Akt/PKB protein in acute myeloid leukemia: its significance as a prognostic variable. Leukemia, 17, 995-997). While the phosphorylation of Thr308 in Akt does not seem to change, the levels of Akt protein are significantly reduced after 6 h of apigenin treatment (FIG. 5). These results suggest that apigenin could mediate two separate responses on Akt, one rapid response involving dephosphorylation of Ser473 and one more slower and sustained effect involving Akt protein degradation. Interestingly, Akt has been reported to be a caspase-3 substrate (Widmann, C., Gibson S. and Johnson G. L. (1998) Caspase-dependent cleavage of signaling proteins during apoptosis. J. Biol. Chem., 273, 7141-7147, Rokudai, S., Fujita N., Hashimoto Y. and Tsuruo T. (2000) Cleavage and inactivation of antiapoptotic Akt/PKB by caspases during apoptosis. J. Cell. Physiol., 182, 290-296). Thus, the observed degradation could be part of a regulatory loop in which the initial (and reversible) inactivation by dephosphorylation of Akt, results in the activation of caspase-3 which then (irreversibly), degrades Akt in lower molecular weight peptides which have less kinase activity and facilitates the entry of cells to apoptosis (Rokudai, S., Fujita N., Hashimoto Y. and Tsuruo T. (2000) Cleavage and inactivation of antiapoptotic Akt/PKB by caspases during apoptosis. J. Cell. Physiol., 182, 290-296, Llorens, F., Miro F. A., Casanas A., Roher N., Garcia L., Plana M., Gomez N. and Itarte E. (2004) Unbalanced activation of ERK1/2 and MEK1/2 in apigenin-induced Hela cell death. Exp. Cell Res., 299, 15-26).

However, the results with the SB203580 inhibitor are unexpected based on a simple model in which apigenin results in the activation of p38, which in turns modulates the de-phosphorylation and degradation of Akt, resulting in apoptosis. The observation that in the presence of SB203580, apigenin-treated cells continue to undergo apoptosis despite the presence of phosphorylated Akt suggests two possible models to explain the action of apigenin (FIG. 8). In the first model, apigenin could be acting on the pathway at two points, one upstream of p38 (resulting in the activation of p38, FIG. 6A), which in turn results in the dephosphorylation and degradation of Akt, and the other downstream of Akt, activating the apoptotic machinery (FIG. 8, left). In the second model (FIG. 8, right), apigenin would be acting downstream of Akt, activating the apoptotic machinery. The activation of apoptosis would result in the positive feedback regulation of the pathway involving p38 and Akt. This feedback regulation would act upstream of p38, explaining how apoptosis continues to happen in the presence of SB203580. There are at least two lines of circumstantial evidence that suggest the possible existence of the proposed feedback loop. First, several kinases and phosphatases are known to be targets for caspases, resulting in either their activation or inactivation (Widmann, C., Gibson S. and Johnson G. L. (1998) Caspase-dependent cleavage of signaling proteins during apoptosis. J. Biol. Chem., 273, 7141-7147, Torres, J., Rodriguez J., Myers M. P., Valiente M., Graves J. D., Tonks N. K. and Pulido R. (2003) Phosphorylation-regulated cleavage of the tumor suppressor PTEN by caspase-3. J. Biol. Chem., 278, 30652-30660). Second, we have recently shown that a member of the PKC (protein kinase C) family interacts with and modulates directly the activity of caspase-3 (Voss, O. H., Kim S., Wewers M. D. and Doseff A. I. (2005) Regulation of monocyte apoptosis by Protein Kinase Cä (PKCä)-dependent phosphorylation of caspase-3. J. Biol. Chem., 10.1074/jbc.M412449200). PKCs have been previously described in some systems to function in the signal transduction pathway upstream of p38 conferring a feedback loop for their regulation has been postulated (Tanaka, Y., Gavrielides M. V., Mitsuuchi Y., Fujii T. and Kazanietz M. G. (2003) Protein kinase C promotes apoptosis in LNCaP prostate cancer cells through activation of p38 MAPK and inhibition of the Akt survival pathway. J. Biol. Chem., 278, 33753-33762, Dempsey, E. C., Newton A. C., Mochly-Rosen D., Fields A. P., Reyland M. E., Insel P. A. and Messing R. O. (2000) Protein kinase C isozymes and the regulation of diverse cell responses. Am. J. Physiol. Lung Cell Mol. Physiol., 279, L429-438, Brodie, C. and Blumberg P. M. (2003) Regulation of cell apoptosis by protein kinase c ä. Apoptosis, 8, 19-27).

Altogether, the studies presented here provide evidence that apigenin is a potent inducer of apoptosis in two myeloblastic leukemia cell lines. Our studies show that the caspase-9/caspase-3 pathway mediates apigenin-induced apoptosis and highlight novel aspects of the signal transduction cascade that participates in the initiation of the apoptotic process by plant metabolites.

### Example 2

### Apigenin Induces Cell Death of Stimulated Monocytes

Apigenin induces cell death on LPS-treated monocytes. Monocytes were stained with calcein AM and PI as described in Material and Methods to evaluate the percentage of cell death and survival. Monocytes freshly isolated (Fresh), treated for 16 h with 10 ng/ml LPS alone, left untreated (NT) or (A) treated with different doses of apigenin, (B) with LPS and apigenin, or (C) with LPS 1 h prior to the addition of apigenin. Values represent the means ±SEM (N=#, *P<0.05 compared to LPS alone).

### Example 3

### Apigenin Induces Reactivation of the Apoptotic Caspase-3 in Stimulated Monocytes

Apigenin reactivates caspase-3 on LPS-stimulated monocytes. Caspase-3 activity was determined by the DEVD-AFC assay in monocytes freshly isolated (Fresh) or monocytes cultured for 18 h left untreated, treated with 10 ng/m 1 LPS or (A) with different doses of apigenin alone, (B) with LPS and different doses of apigenin or (C) with LPS 1 h prior to the addition of apigenin. Values represents means ±SEM (N=3, * P<0.05, compared to LPS alone).

### Example 4

### Apigenin Inhibits the Release of Inflamatory IL-1B in Inflammatory Monocytes

Effect of apigenin on IL-1β release. IL-1β released was determined by sandwich ELISA in supernatants of freshly isolated monocytes, or monocytes cultured for 18 h left untreated, treated with 10 ng/ml of LPS or A with different doses of apigenin. B. LPS and different doses of apigenin added at the same time. C. LPS added 1 h prior to the addition of different doses of apigenin. Values represent means ±SEM (N=5, * P<0.05, compared to LPS alone)

### Example 5

### Apigenin Inhibits Expression of Pro-Inflammatory Cytokines

Apigenin inhibits the expression of inflammatory cytokines. Expression of IL-1β, IL-8 and TNFα was analyzed by quantitative PCR using lysates from monocytes left untreated, treated with 10 ng/ml of LPS or with LPS and 10 μM of apigenin. Values represent means ±SEM (N=4).

1. A method for treating inflammation in a subject in need of the same, the method comprising administering to the subject apigenin, an apigenin derivative, apigenin and at least one apigenin derivative, or a combination of apigenin derivatives.

2. The method of claim 1, wherein the subject has a chronic inflammatory disease.

3. The method of claim 1, wherein the subject has an inflammatory disease or condition selected from an autoimmune disease, arthritis, sarcoidosis, sepsis, atherosclerosis, and pulmonary fibrosis.

4. The method of claim 1 wherein the subject is a mammal.

5. The method of claim 4, wherein the subject is a human subject.

6. The method of claim 1, wherein at least one apigenin derivative chosen from a naturally occurring derivative of apigenin, an apigenin salt, an apigenin ester, a monocyte apoptosis-inducing metabolite of apigenin, and a synthetic derivative of apigenin is administered to the subject.

7. The method of claim 1, wherein at least one synthetic apigenin derivative is administered to the subject, in which the hydroxyl group attached to C-7 and/or C-5 in the A ring and/or the hydroxyl group attached to C-4′ in the B ring are glycosylated or acylated or replaced with an amino group or halogens (e.g., Cl) and/or by the addition of nitro or amino groups at position 5′ in the B ring.

8. Use of apigenin, an apigenin derivative, apigenin and an apigenin derivative, or a combination of apigenin derivatives, in the preparation of a medicament for use in the treatment of inflammation.

9. Use of apigenin, an apigenin derivative, apigenin and an apigenin derivative, or a combination of apigenin derivatives, in the preparation of a medicament for use in the treatment of a chronic inflammatory disease or condition.

10. The use according to claim 10, wherein the chronic inflammatory disease or condition is chosen from an autoimmune disease, arthritis, sarcoidosis, and sepsis.

11. Use of apigenin, an apigenin derivative, apigenin and an apigenin derivative, or a combination of apigenin derivatives, in the preparation of a medicament for use in the treatment of acute monocytic leukemia.

12. The use according to claim 9, 10, or 11, wherein the apigenin derivative is a synthetic derivative in which the hydroxyl group attached to C-7 and/or C-5 in the A ring and/or the hydroxyl group attached to C-4′ in the B ring are glycosylated or acylated or replaced with an amino group or halogen (e.g., Cl) and/or by the addition of nitro or amino groups at position 5′ in the B ring.

13. A pharmaceutical composition comprising an apigenin derivative and an excipient wherein the apigenin derivative is a synthetic derivative in which the hydroxyl group attached to C-7 and/or C-5 in the A ring and/or the hydroxyl group attached to C-4′ in the B ring are glycosylated or acylated or replaced with an amino group or halogens (e.g., Cl) and/or by the addition of nitro or amino groups at position 5′ in the B ring.

14. A method for treating acute monocytic leukemia in a subject in need of the same, the method comprising administering to the subject apigenin, an apigenin derivative, apigenin and at least one apigenin derivative, or a combination of apigenin derivatives.
